(19) United States
(12) Patent Application Publication
(10) Pub. No.: US 2023/0104918 A1
(43) Pub. Date: Apr. 6, 2023

(54) CONNECTION SYSTEM FOR TRANSMITTING ENERGY AND/OR DATA FROM AND/OR TO AN IMPLANTABLE BLOOD PUMP, AND VENTRICULAR ASSIST DEVICE

(71) Applicant: Berlin Heart GmbH, Berlin (DE)

(72) Inventors: Konstantin Austilat, Berlin (DE); Christian Robbenmenke, Berlin (DE)

(73) Assignee: Berlin Heart GmbH, Berlin (DE)

(21) Appl. No.: 18/064,559

(22) Filed: Dec. 12, 2022

Related U.S. Application Data
(63) Continuation of application No. PCT/EP2021/062859, filed on May 14, 2021.

(30) Foreign Application Priority Data
Jun. 11, 2020 (EP) EP20179453.4

(51) Int. Cl. A61M 60/878; A61M 60/178; A61M 60/422; A61M 60/216

(52) U.S. Cl. CPC A61M 60/878; A61M 60/178; A61M 60/422; A61M 60/216

(57) ABSTRACT

The application relates to a connection system for transmitting energy and/or data from and/or to an implantable blood pump. The proposed connection system comprises a first connection unit, which can be or is connected to the blood pump, and a second connection unit, which can be or is connected to a control and/or energy unit. In particular, the first connection unit can be connected to the blood pump by means of an implantable line. The second connection unit can be connected to the control and/or energy unit by means of a transcutaneous line. The first connection unit and the second connection unit can be wirelessly coupled to each other for wireless transmission of energy and/or data. Furthermore, the first connection unit and the second connection unit are implantable, so that both the first connection unit and the second connection unit are designed for use within the body of a patient.

### PRIORITY

This application claims priority as a Continuation of PCT/EP2021/062859, filed on May 14, 2021, entitled “CONNECTION SYSTEM FOR TRANSMITTING ENERGY AND/OR DATA FROM AND/OR TO AN IMPLANTABLE BLOOD PUMP, AND VENTRICULAR ASSIST DEVICE”, published as WO 2021/249721, which claims priority to European Patent Office Application No. EP 20179453.4 filed on Jun. 11, 2020. The entire disclosure of the above applications are incorporated herein by reference.

### TECHNICAL FIELD

The present application is in the field of medical engineering and in particular in the field of implantable blood pumps for assisting cardiac function. The application relates to a connection system for transmitting energy and/or data from and/or to an implantable blood pump and a cardiac assist system comprising such a connection system.

### BACKGROUND

Cardiac assist systems with implantable blood pumps are known from the prior art. These cardiac assist systems can be used when the cardiac function of a patient has to be assisted or replaced. Current systems that are used in this context are so-called VADs (ventricular assist devices). Such systems can be embodied for example as a so-called LVAD (left ventricular assist device), RVAD (right ventricular assist device) or BiVAD (bi-ventricular assist device). Besides the implantable blood pump, which is implanted in the patient during operation, these systems also generally comprise a control unit, which for example is arranged outside the patient's body and is connected to the blood pump via a transcutaneous line (driveline). Data can be exchanged between the blood pump and the control unit via the transcutaneous line. In addition, an energy unit arranged outside the body is generally provided, which is likewise connected via the transcutaneous line to the blood pump and via which the blood pump is supplied with electrical energy. The blood pump generally comprises a motor with a stator and a rotor which is provided with a blading and which is arranged in a flow channel of the blood pump. The motor of the blood pump can be driven by energy delivered from the control units, for example as the result of a current flow being generated in windings of the stator, which sets the rotor together with its blading in rotation in order to convey the patient's blood.

### SUMMARY

Since the transcutaneous line, which is generally embodied as a partially implantable cable, is passed through the patient's skin, a point of passage is required. The point of passage is disadvantageously susceptible to infection. In order to avoid points of passage, it is conceivable to provide connection systems by which a contactless transmission of energy or data through the skin is possible. So-called TET (Transcutaneous Energy Transfer) systems of this kind generally provide an implanted coil, which is connected to the blood pump via a cable. In addition, an extracorporeal coil is provided, which is inductively coupled to the implanted coil. This extracorporeal coil is arranged outside the body and is connected to the energy and the control unit via a line arranged fully outside the patient's body. This kind of transcutaneous energy transmission, however, has only a low level of efficiency, causing high heating of the surrounding tissue. In addition, the implanted coil and the extracorporeal coil necessarily have to be placed very precisely relative to each other in order to ensure a reliable transmission. Systems of this kind, in order to ensure fail-safety, additionally require an implantable battery, which contributes to a relatively high level of complexity of systems of this kind.

Further approaches are also conceivable, by means of which complications relating to the above-described infections of the point of passage are mitigated. For example, it is conceivable to equip the driveline, which connects the extracorporeal control and energy units to the implantable blood pump, with an implanted plug connector. This would have the advantage that, in the event of an infection of the point of passage, only a short cable portion between the energy and control unit and the implantable plug connector would have to be replaced, and the blood pump in this case could remain in the patient. Thus, only a relatively minor surgical intervention would be necessary. In addition, with this solution it is possible to dispense with the implanted battery and the disadvantages associated therewith. Implantable plug connectors, however, are relatively difficult to produce from a technical viewpoint. Above all, the impermeability, susceptibility to corrosion, and the installation of the conductors are technical problems here that present challenges.

Against the background of the above-described aspects, it is an object of the present application to propose an improved connection system for transmitting energy and/or data from and/or to an implantable blood pump, as well as a correspondingly improved cardiac assist system. In particular, an object of the present application is to propose a connection system that allows a cardiac assist system of relatively simple construction, with which problems occurring in relation to infections of the point of passage through the skin are mitigated. In addition, the risk of tissue damage in comparison to the above-described prior art is intended to be kept low.

These objects are achieved by a connection system having the features of independent claim 1 and by a cardiac assist system having the features of a further claim. Advantageous developments are provided by the features of the dependent claims and the example embodiments.

The proposed connection system for transmitting energy and/or data from and/or to an implantable blood pump comprises a first connection unit, which can be connected or is connected to the blood pump,

and a second connection unit, which can be connected or is connected to a control and/or energy unit. The first connection unit can be connected to the blood pump in particular by means of an implantable line. The second connection unit can be connected to the control and/or energy unit by means of a transcutaneous line. The transcutaneous line can be implantable fully or at least in a portion that is to be implanted, and in particular on its outer side can comprise biocompatible material or can consist of biocompatible material. The first connection unit and the second connection unit can be wirelessly coupled to each other for wireless transmission of energy and/or data. In addition, the first connection unit and the second connection unit are implantable, so that both the first connection unit and the second connection unit are designed for use within the body of a patient. The control and/or energy unit is typically of extracorporeal design. However, it can be provided in other embodiments that the control and/or energy unit is implanted or implantable. For example, the control and/or energy unit can be a unit (in particular implanted or implantable) which is connected fixedly or releasably to the second connection unit of the connection system. In some embodiments, the control and/or energy unit can be embodied such that it is accommodated together with the second connection unit in a single housing, for example as an implanted control unit with integrated interface for wireless coupling to the first connection unit, which can be connected or is connected to the pump.

The present application also relates to a cardiac assist system comprising a connection system as described above or below. Furthermore, the cardiac assist system can comprise the blood pump and/or the extracorporeal control and/or energy unit. The first connection unit can be connectable to the blood pump by means of the implantable or implanted line. The second connection unit can be connectable, in particular releasably, to the extracorporeal control and/or energy unit via the fully or at least partially implantable transcutaneous line. With use of the cardiac assist system, the transcutaneous line generally passes through a point of passage in the skin.

In the event that an infection of the point of passage occurs, the proposed connection system allows for simple replacement of only the transcutaneous line and the second connection unit. Compared to implantable plug connectors, the proposed connection system has the advantage that the connection units can be fully encapsulated. Thus, problems that occur with plug connectors in relation to soiling and corrosion of the electrical plug contacts as well as problems arising generally in relation to incoming liquid, such as short circuits, are remedied by the proposed connection system. In particular, it can be provided that the connection system for coupling the connection units to each other, when the transcutaneous line and the fully implanted line for connection of the blood pump to the connection system are connected, has no external, in particular no active, electrical conductors, in particular plug contacts. In addition, problems that may occur in other systems in relation to the sealing and insulation of external plug contacts are avoided by the proposed connection system.

In comparison to transcutaneous energy transmission systems (TET), the proposed connection system allows significantly increased levels of efficiency. This is achieved by arranging the connection units very closely, in particular with contact, during their use. Problems in relation to coupling factors reduced by the distance for example of a transmitter and a receiver coil from each other and reduced efficiency are thus avoided. In particular, thermal tissue stress and/or tissue damage occurring as a result of reduced efficiency is avoided by means of the proposed connection system. Additionally, as a result of the proposed connection system, the cardiac assist system does not necessarily need to have an implanted battery for an emergency supply.

In a method which may likewise be a subject of the present application, a cardiac assist system as described above or below is firstly provided. In particular, once an infection of the point of passage has been determined, the second connection unit and the transcutaneous line can be removed, as necessary following a disconnection of the first connection unit, and can be replaced for a further second connection unit and a further transcutaneous line. Here, it can be provided that the transcutaneous line is disconnected from the extracorporeal control and/or energy unit and that the further transcutaneous line is connected to the extracorporeal control and/or energy unit.

It can be provided that the first and second connection unit can be wirelessly coupled to each other inductively and/or capacitively. For example, the first connection unit can have at least one coil. In addition, the second connection unit can have at least one coil. The first connection unit and the second connection unit can be coupled to each other, in particular inductively, via the coils for wireless transmission of energy and/or data.

The term “implantable” and the fact of being designed for use within the body of a patient shall be understood by a person skilled in the art to be structurally limiting within the context of the present application. For example, a person skilled in the art of medical engineering will know what requirements are placed on implantable devices from a structural viewpoint.

The first connection unit and the second connection unit are generally accommodated in separate housings. It can be provided that the first connection unit has a housing and that the second connection unit has a housing, wherein the housing of the first connection unit and/or the housing of the second connection unit are liquid-tight, in particular impervious to bodily fluids, for example blood-tight. In some embodiments, the housings are structurally identical. Since the connection units are intended for implantation, they have a space-saving design. In particular, it can be provided that the connection units are substantially free from voids and/or that the housings do not contain any air pockets. For example, the coil of the first connection unit is accommodated in the housing of the first connection unit, and for example the coil of the second connection unit is accommodated in the housing of the second connection unit. The housing of the first connection unit and/or the housing of the second connection unit is typically biocompatible. In particular, the housing of the first connection unit and/or the housing of the second connection unit can have a completely biocompatible outer surface. The connection of the transcutaneous line to the second connection unit and/or the connection of the implantable line for connection of the first connection unit the blood pump is generally likewise liquid-tight and/or implantable.

It can be provided that the connection system has a mechanical connection means, by which the first connection unit and the second connection unit can be connected releasably to each other, in particular can be fastened to each other non-displaceably and/or rigidly. Due to the mechanical connection means, a mechanical fixing of the connection units can be achieved, so that the connection units are held in position for reliable energy and/or data transmission and so that an unintentional separation within the patient's body is prevented. In order to prevent unintentional separation, it can be provided that the mechanical connection means establishes between the first connection unit and the second connection unit a connection requiring an action of force of at least 10 N, in particular at least 15 N in order to be released. It can be provided that the mechanical connection means is a screw closure, a bayonet closure, a detent closure and/or a snap-action closure.

It can also be provided that the mechanical connection means is an external housing which is designed to accommodate the first connection unit and the second connection unit. The external housing can be placed around both connection units, for example once the connection units have been oriented relative to each other. This has the advantage, that when replacing one side, i.e. one connection unit, both sides are freely accessible with the removal of the external housing and the replacement is thus easier. If the external housing comprises, for example, silicone or a silicone coating for both coils, an in-growth is additionally avoided, which likewise allows for easier replacement.

It can be provided that the connection system has a mechanical orientation means. By way of the mechanical orientation means, the first connection unit and the second connection unit can be orientable relative to each other so that the first connection unit and the second connection unit come to lie in a predefined orientation, in particular position and arrangement, relative to each other. The orientation means can ensure, for example, that the first connection unit and the second connection unit are oriented such that the optionally provided coils come to lie in a desired orientation relative to each other when the first connection unit is arranged relative to the second connection unit. Undesirable lateral deviations and the positioning of the connection units relative to each other, as may occur for example in TET systems, can thus be prevented.

The connection system is arranged here typically, in particular fully, in a skin pocket and/or in the subcutis of the patient. However, it can also be provided that the connection system is placed, in particular fully, beneath the superficial fascia. In this way, the connection system can make use of the effect of the fascia as an additional, natural barrier against infection. The connection system can be formed in particular in respect of its spatial-physical properties in such a way that it is designed and suitable for arrangement in particular fully in the subcutis or beneath the superficial fascia of the patient. The transcutaneous line, for this purpose, can have a correspondingly suitable minimum length. However, the transcutaneous line is typically not longer than 1 m. In the event of an infection of the point of passage, in order to prevent this infection from reaching the connection system before it is discovered and treated, a sufficient distance can be provided between the point of passage and the connection system. For example, a distance between the point of passage and the connection system can be at least 5 cm, in particular at least 8 cm. So that, however, there is no need to replace an unnecessarily long length of the transcutaneous line, the distance between the point of passage and the connection system can be at most 20 cm, in particular at most 12 cm.

The connection system is generally designed to wirelessly transmit a power of at least 2 W, in particular at least 4 W or 5 W. It can be provided that the connection system is designed to transmit a power of at most 30 watts. In this way, the connection system is particularly suitable for energy transmission from the energy unit the blood pump. Here, the proposed connection system is particularly suitable, since, during its use, skin layers which might otherwise be thermally damaged during the transmission of the desired powers are not necessarily arranged between the connection units.

Since the proposed connection system allows a shorter distance between the connection units, it is possible to use small overall sizes for reliable energy and/or data transmission. Since, for example, only relatively thin housing walls of the housings of the connection units are required, a distance between the optionally provided coils of the connection units, in particular during use of the connection system, can be less than 4 mm, in particular less than 2 mm. Due to the associated high efficiency of the proposed connection system, small overall sizes can be achieved, for example by using flat components and/or by selecting small coil diameters of the coils of the connection units. For example, the first connection unit and the second connection unit can have a flat design. The first connection unit and the second connection unit, when these rest against each other with contact, can have a total height of less than 20 mm, in particular less than 10 mm. in particular due to a short height in comparison to the extents in other directions, the connection system is suitable for placement beneath the skin of the patient, in particular in the subcutis and/or beneath the superficial fascia. In addition, it can be provided that the first connection unit and/or the second connection unit have a diameter of at most 40 mm, in particular at most 30 mm. In particular, a maximum housing diameter of the first connection unit and/or of the second connection unit can be less than 30 mm. The advantage of the small overall size comes into effect in that the coils, with more reliable energy and data transmission as well as less tissue damage, have a diameter which can be smaller than the diameter in known TET systems. A diameter of the coil of the first connection unit and/or a diameter of the coil of the second connection unit can be less than 40 mm, in particular less than 30 mm, for example less than 20 mm. The coils are generally oriented in such a way that they are positioned with a coil plane along a plane of a planar extent of the connection units.

If the system provides, during implantation, that the second connection unit, in particular an inductive plug connector, must be guided through the skin, it is advantageous if an outer cross section is small so that an incision into the skin remains small. Alternatively to a flat embodiment of the plug connector, a round connector can thus also be provided. If the round connector has a coil for inductive data transmission, this coil is generally arranged substantially transversely to a longitudinal direction of the transcutaneous line. A second connection unit formed as a round connector, in particular with relatively small outer cross section of at most 20 mm, can be guided through the skin and for example mechanically connected to the first connection unit by means of methods described above or below. The first connection unit can also be formed accordingly. It can be provided that this is also passed through the skin, specifically in the direction of the control unit.

In some embodiments, the first connection unit and/or the second connection unit can have a rounded design in respect of their components that are to be brought into connection with the body. In this way, the first connection unit and the second connection unit are particularly suitable for use within the body of the patient. For example, housings of the first connection unit and/or the second connection unit can be formed without steps and/or edges on their outer side.

In some embodiments, the first connection unit and/or the second connection unit is deformable, in particular elastically deformable and/or flexible. In this way, the connection units are particularly suitable for implantation into the skin layers of the patient described above and below.

It can furthermore be provided that the first connection unit and/or the second connection unit comprises a material that is both biocompatible and also flexible, in particular silicone. In particular, the optionally provided coils of the first and/or second connection units can be surrounded by this material, in particular embedded in and/or overmoulded by said material.

If an individual direct voltage or alternating voltage is transmitted to the blood pump, this is generally carried out using a coil pair, i.e. a first and a second connection unit. This requires a generation of the motor phase voltages within the blood pump. If the motor phase voltages, typically 3 phases, are to be transmitted from the external control unit to the blood pump, 3 plug connectors are generally also necessary. These can be divided intra-corporeally after having passed through the point of passage through the skin, so that 3 plug connectors are placed individually. The three plug connectors for the motor phases can also be accommodated in a common housing and arranged so that, geometrically, they occupy only a small amount of space. For example, it is thus also possible that more than 3 signals are transmitted to the blood pump.

The application can relate to a first connection unit and/or second connection unit having the properties described above or below.

Example embodiments will be described hereinafter with reference to the drawings.

### BRIEF DESCRIPTION OF THE DRAWINGS

FIG. 1 shows a schematic view of a cardiac assist system comprising a connection system.

FIG. 2 shows an arrangement of the connection system beneath the skin of a patient.

FIG. 3(a) shows one view of the connection system.

FIG. 3(b) shows another view of the connection system.

FIG. 3(c) shows another view of the connection system.

FIG. 4 shows a plan view of coils of a connection unit of the connection system.

### DETAILED DESCRIPTION

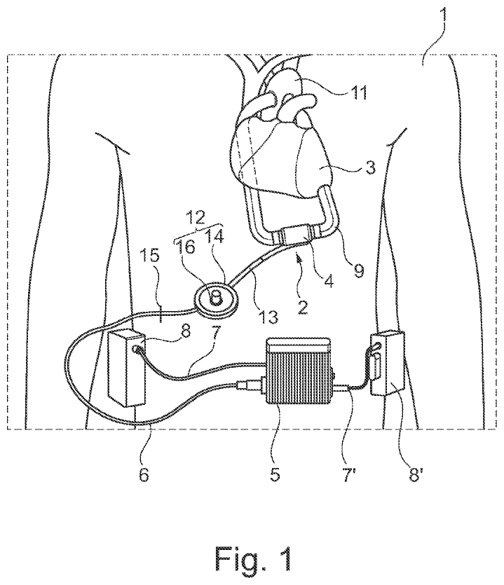

FIG. 1 shows schematically a body 1 of the patient, in which a blood pump 2 for assisting the function of the heart 3 is implanted. The blood pump 2 is formed as part of a cardiac assist system and has a motor which is typically embodied as an electric motor with a rotor and which is received in a biocompatible pump housing 4 of the blood pump 2. The pump housing 4 is connected to a control unit 5, which is arranged outside the patient's body and is connected to the blood pump 2 via a transcutaneous line 6. The control unit 5 is additionally connected via two cables 7, 7′ to an energy unit formed as a pair of extracorporeal batteries 8, 8′. The pump housing 4 is connected to an inlet channel 9, via which blood is removed from a chamber of the heart 3, and to a cannula 10, via which blood can be conveyed into a blood vessel 11.

The control unit 5 is designed to actuate the motor of the blood pump 2 to convey the blood and to supply said motor with energy. In addition, sensor data for example can be transmitted from the blood pump 2 to the control unit 5. For this purpose, the transcutaneous line 6 is connected via an implanted connection system 12, which is arranged in particular fully within the patient's body, to a fully implanted cable 13. The fully implanted cable 13 connects a first connection unit 14 of the connection system 12 to the blood pump 2. The transcutaneous cable 6 passes through the patient's skin at a point of passage 15, i.e. at an opening in the patient's skin, and connects the control unit 15 to a second connection unit 16 of the connection system 12. In order to transmit data and energy with a power of more than 2 W, in particular more than 5 W, the first connection unit 14 is coupled wirelessly to the second connection unit 16. In the shown examples, an inductive coupling is provided, however, in other embodiments a capacitive coupling can also be provided. There are generally no current-conducting connections between the first connection unit and the second connection unit.

Typically, the connection system 12 is designed to transmit electrical energy, for power supply of the blood pump 2, from the second connection unit 16 to the first connection unit 14. In addition, the connection system 12 is generally designed to transmit electrical signals, for example sensor data or control data, both from the second connection unit 16 to the first connection unit 14 and from the first connection unit 14 to the second connection unit 16. Here, the bodily fluid of the patient can be considered to be a disturbance variable, since the connection system 12 is located fully in the patient's body.

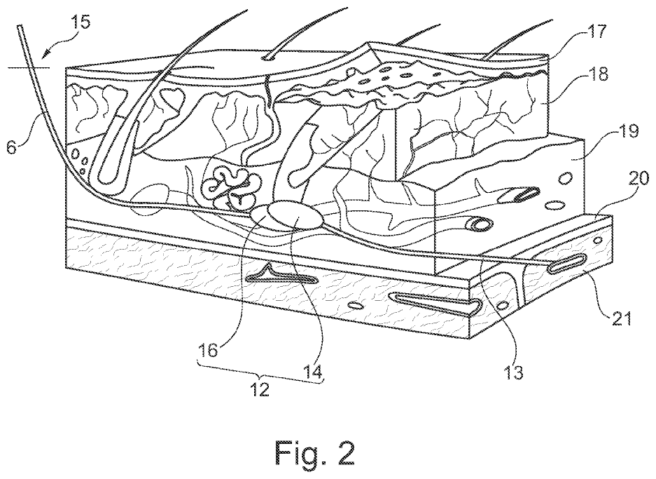

FIG. 2 shows an arrangement of the connection system 12 beneath the patient's skin. Recurring features are provided in this figure and in the following figures with the same reference signs. Shown is the structure of the body layers and in particular skin layers, comprising the epidermis 17, the dermis 18, the subcutis 19, the fascia 20, and a muscle 21. The connection system 12, during use of the blood pump 2 or of the cardiac assist system, is generally arranged at a distance of approximately 8 to 12 cm from the point of passage 15. The connection system 12 with the connection units 14, 16 is of flat design and arranged such that the direction of its planar extent lies in a layer plane of the body layer. The connection system 12 can be implanted in a skin pocket of the patient. In the shown example, the connection system 12 is arranged in the subcutis, so that the connection system is relatively easily accessible for replacement of the second connection unit 16 and of the transcutaneous line 6 in the case of an infection of the point of passage 15. However, it can also be provided that the connection system 12 is arranged beneath the superficial fascia, so that this forms an additional barrier against infection.

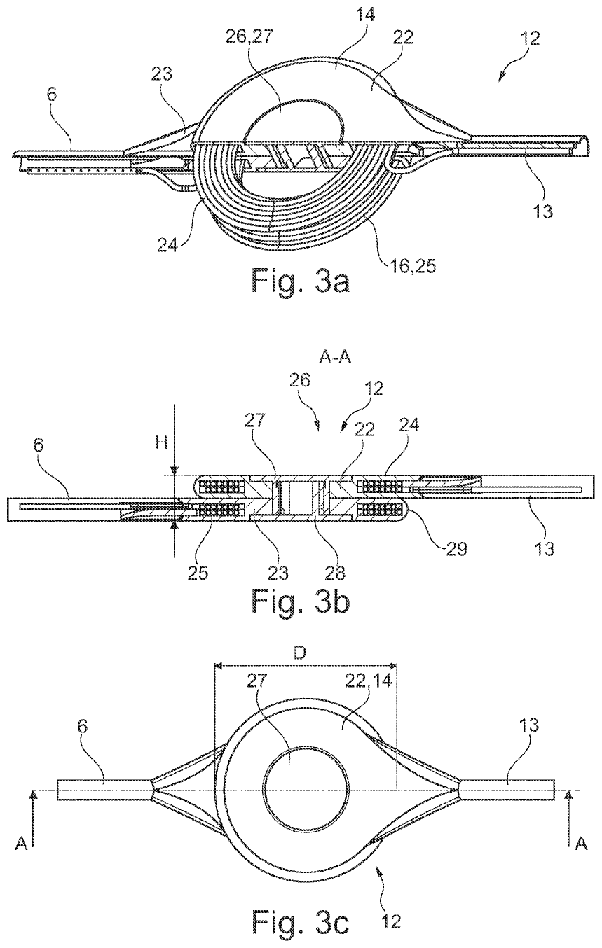

FIGS. 3(a) to (c) show different views of the connection system 12. The first connection unit 14 and the second connection unit 16 have a housing 22, 23 respectively. Parts of the housing 22, 23 are not shown in FIG. 3(a) in order to show coils 24, 25 accommodated in said housings. One of the coils 24 is to be assigned to the first connection unit 14, whereas a second of the coils 25 is to be assigned to the second connection unit 16. The coils 24, 25 are arranged concentrically one above the other for energy and/or data transmission. In the shown example, the coils are formed without a core. Generally, the coils 24, 25 can be, but do not necessarily have to be, of the same size. In some embodiments a ferrite plate is integrated in order to increase the efficiency.

As shown in a sectional view in FIG. 3(b), for energy and/or data transmission, the housings 22, 23 of the first and second connection unit 14, 16 lie against each other with contact during use of the connection system 12. The housings 22, 23 are each formed by a biocompatible, elastically deformable silicone body, by which the coils 24, 25 respectively are overmoulded. The housings 22, 23 enclose the coils 24, 25 generally in a manner impervious to bodily fluids and are manufactured from a material resistant to corrosion and hydrolysis, in this case silicone. The housings are free of sharp edges and have a rounded outer side, see for example the end of the second connection unit 16 denoted by reference sign 29. Apart from the lines 6, 13, no parts of the cardiac assist system protrude from the connection units 14, 16.

A height H of the entire connection system 12 is less than 10 mm when the connection units 14, 16 rest against each other with contact, so that the connection system 12 requires only a small implantation volume. For efficient inductive coupling, a vertical distance of the coils 24, 25 is less than 2 mm when the connection units 14, 16 rest against each other with contact.

The connection units 14, 16 are additionally mechanically connected to each other and oriented relative to each other so that the coils 24, 25, as shown in FIG. 3(a) to (c), come to lie parallel and concentrically in a defined position relative to each other. For this purpose, the connection system 12 has a mechanical connection means 26, which at the same time serves as an orientation means. In the shown embodiment, the connection means 26 is formed by a detent connection comprising a pair 27, 28 of detent elements engaging in each other. The connection means 26 is formed such that it also holds reliably in the body, wherein an action of force of more than 10 N, for example 20 N can be necessary to release the connection. In respect of the connection means, it can be provided for example that the connection units 14, 16 are secured via threaded sleeves (connection units are screwed to each other), which would be suitable in particular in the case of a hard housing material. Alternatively, a bayonet closure is also a possible connection means.

FIG. 3(c) shows a plan view of the connection system 12. A diameter D of the coils 24, 25 may for example amount to relatively small 10 or 15 mm. A diameter of the housings 22, 23 is only negligibly larger. In some embodiments, the connection system 12 has a kink protection means at the transition between the line 6 or 13 and the connection unit 14 or 16 respectively in order to prevent a kinking of the lines. A strain relief element and/or a shielding transition can also be provided between the line 6 or 13 and the connection unit 14. In the shown example, the kink protection means is formed by ends of the elastic silicone housing 22, 23 facing the lines 6, 13.

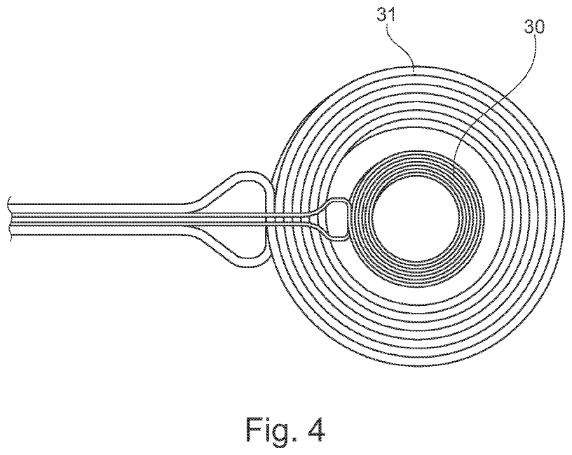

FIG. 4 shows a pair of concentrically arranged coils 30, 31 according to a further embodiment, which, as described above, can be provided in the housing 22 of the first connection unit 14. In this case, the coil with the reference signs 31 forms a primary coil for energy transmission. The smaller coil with the reference signs 30 forms a secondary coil for simultaneous signal transmission. The second connection unit 16 can have a primary coil and a secondary coil correspondingly.

Features of the various designs disclosed in the example embodiments can be combined with each other and claimed individually.

The application relates, amongst other things, to the following embodiments:

A connection system for transmitting energy and/or data from and/or to an implantable blood pump, the connection system comprising a first connection unit, which can be connected to the blood pump, and a second connection unit, which can be connected to an extracorporeal control and/or energy unit, wherein the first connection unit and the second connection unit can be wirelessly coupled to each other for wireless transmission of energy and/or data, wherein the first connection unit and the second connection unit are implantable, so that both the first connection unit and the second connection unit are designed for use within the body of a patient. The first connection unit has at least one coil and the second connection unit has at least one coil, wherein the first connection unit and the second connection unit can be inductively coupled to each other via the coils for wireless transmission of energy and/or data. The first connection unit has a housing and the second connection unit has a housing, wherein the housing of the first connection unit and the housing of the second connection unit are liquid-tight, in particular impervious to bodily fluids. A mechanical connection means by which the first connection unit and the second connection unit can be releasably connected to each other. The mechanical connection means establishes between the first connection unit and the second connection unit a connection requiring an action a force of at least 10 N, in particular at least 15 N in order to be released. The mechanical connection means is a screw closure, a bayonet closure, a detent closure and/or a snap-action closure. There is a mechanical orientation means, by which the first connection unit and the second connection unit can be oriented relative to each other so that the first connection unit and the second connection unit come to lie in a predefined orientation relative to each other. The connection system is designed to transmit wirelessly a power of at least 2 W, in particular at least 4 W. The first connection unit and the second connection unit have a flat design. The first connection unit and the second connection unit, when these rest against each other with contact, have a total height of less than 20 mm, in particular less than 10 mm. The first connection unit and the second connection unit have a diameter of at most 40 mm, in particular at most 30 mm. The first connection unit and the second connection unit have a rounded design in their components that are to be brought into connection with the body. The first connection unit and/or the second connection unit is deformable. The first connection unit and/or the second connection unit comprises silicone.

In another embodiment, a cardiac assist system includes a connection system according to one of the preceding embodiments and furthermore includes the blood pump and/or the extracorporeal control and/or energy unit.

1. A connection system for transmitting energy and/or data to or from an implantable blood pump, the connection system comprising:
a first connection unit connected to the blood pump;
a second connection unit connected to a control and/or energy unit, wherein the first connection unit and the second connection unit are wirelessly coupled for wireless transmission of energy and/or data, further wherein the first connection unit and the second connection unit are implantable, so that both the first connection unit and the second connection unit are designed for use within a body of a patient.

2. The connection system according to claim 1, wherein the first connection unit has at least one coil and the second connection unit has at least one coil, wherein the first connection unit and the second connection unit can be inductively coupled to each other via the coils for wireless transmission of energy and/or data.

3. The connection system according to claim 1, wherein the first connection unit has a housing, and the second connection unit has a housing, wherein the housing of the first connection unit and the housing of the second connection unit are liquid-tight and impervious to bodily fluids.

4. The connection system according to claim 1, further comprising:
a mechanical connection means by which the first connection unit and the second connection unit can be releasably connected to each other.

5. The connection system according to claim 4, wherein the mechanical connection means establishes a connection between the first connection unit and the second connection unit requiring an action of force of at least 10 N in order to be released.

6. The connection system according to claim 4, wherein the mechanical connection means is a screw closure, a bayonet closure, a detent closure, or a snap-action closure.

7. The connection system according to claim 1, further comprising:
a mechanical orientation means, by which the first connection unit and the second connection unit can be oriented relative to each other so that the first connection unit and the second connection unit come to lie in a predefined orientation relative to each other.

8. The connection system according to claim 1, wherein the connection system is designed to transmit wirelessly a power of at least 2 W.

9. The connection system according to claim 1, wherein the first connection unit and the second connection unit have a flat design.

10. The connection system according to claim 1, wherein the first connection unit and the second connection unit have a total height of less than 20 mm when the connection units are in contact.

11. The connection system according to claim 1, wherein the first connection unit and the second connection unit have a diameter of at most 40 mm.

12. The connection system according to claim 1, wherein the first connection unit and the second connection unit have a rounded design for any components that are to be brought into connection with the body.

13. The connection system according to claim 1, wherein the first connection unit and/or the second connection unit is deformable.

14. The connection system according to claim 13, wherein the first connection unit and/or the second connection unit comprises silicone.

15. The connection system according to claim 1, wherein the control and/or energy unit is extracorporeal.

16. A cardiac assist system comprising
the connection system according to claim 1;
the blood pump; and
the control and/or energy unit.
